(19) United States
(12) Patent Application Publication
(10) Pub. No.: US 2026/0315032 A1
(43) Pub. Date: Oct. 15, 2026

(54) METHOD FOR ULTRASOUND IMAGING OF A MICROVASCULAR STRUCTURE

(71) Applicants: SORBONNE UNIVERSITE, PARIS (FR); CENTRE NATIONAL DE LA RECHERCHE SCIENTIFIQUE, Paris (FR); INSTITUT NATIONAL DE LA SANTE ET DE LA RECHERCHE MEDICALE, Paris (FR); ASSISTANCE PUBLIQUE HOPITAUX DE PARIS, Paris (FR); UNIVERSITE PARIS CITE, PARIS (FR)

(72) Inventors: Olivier COUTURE, PARIS (FR); Vincent HINGOT, PARIS (FR); Louise DENIS, PARIS (FR); Abderrahmane AISSANI, PARIS (FR); Sylvain BODARD, PARIS (FR); Jean-Michel CORREAS, PARIS (FR)

(21) Appl. No.: 19/117,031

(22) PCT Filed: Sep. 29, 2022
(86) PCT No.: PCT/FR2022/051843

§ 371 Date: Apr. 14, 2026

(51) Int. Cl. A61B 8/00; A61B 8/08

(52) U.S. Cl. CPC A61B 8/481; A61B 8/085; A61B 8/461; A61B 8/5223

(57) ABSTRACT

The present invention relates to a method for ultrasound imaging of a microvascular structure, characterized in that it comprises: a step of injecting ultrasound contrast agents into blood vessels in a region comprising a microvascular structure, a step of ultrasound imaging said region comprising the microvascular structure while the contrast agents flow through the blood vessels, the concentration of the contrast agents and/or the ultrasound frequency of the imaging being chosen such that the contrast agents are sufficiently separated to prevent feedback, a step of detecting/filtering and locating the contrast agents, a step of individually monitoring the contrast agents, a step of classifying the behaviors of the individually monitored contrast agents according to at least one predetermined behavior characteristic of contrast agents flowing through the microvascular structure, and a step of imaging the microvascular structure according to the thus-performed classification.

### CROSS-REFERENCE TO RELATED APPLICATION

The present application is a filing under 35 U.S.C. 371 as the National Stage of International Application No. PCT/FR2022/051843, filed Sep. 29, 2022, entitled “METHOD FOR ULTRASOUND IMAGING OF A MICROVASCULAR STRUCTURE,” which is incorporated herein by reference in its entirety for all purposes.

The object of the present invention is a method for ultrasound imaging of a microvascular structure, and in particular of a functional unit of an organ of the human or animal body or a physiological or pathological process affecting the microcirculation. The method notably enables imaging of kidney glomeruli. The object of the invention is also a device for implementing the method.

One of the basic functional units of the kidney is the glomerulus. This component of the nephron is a tangle of capillaries located between two resistance vessels (afferent arteriole and efferent arteriole) which provides its filtration capacity. The glomerulus is affected by a number of chronic diseases such as hypertension, diabetes, autoimmune diseases and cancer which can lead to its complete destruction and therefore the loss of its filtration functions. Glomerular imaging is necessary for the exploration, diagnosis and understanding of these pathologies. However, the diameter of glomeruli in humans is about 200 microns, which is well below the resolution limit of most medical imaging techniques, and their function is only assessed indirectly in the clinic, by blood or urine tests that give access to the overall glomerular filtration rate and not to the observation of individual glomeruli. Renal biopsy is the only current means of observing and analyzing these structures.

In the same way, numerous functional structures have thus been cataloged throughout the human body, wherein the architecture of the microvasculature is closely linked to the function of the organ. For example, the islets of Langerhans and insulin secretion in the pancreas, Kupffer cells and filtration in the liver. For all these structures, medical imaging is limited to the observation of macroscopic processes with poor resolution, which does not provide an understanding of the processes involved, nor good diagnostic tools.

Until recently, imaging technologies for microvascular anatomy and physiology have been limited by two contradictory aspects: resolution and penetration. Indeed, medical imaging techniques such as MRI, CT and ultrasound are unable to visualize the microcirculation directly, as their resolution is generally limited to the millimeter or submillimeter scale (a few hundred microns or more). Indirect techniques, for example the perfusion of the organ by contrast agents, can be used to characterize organ perfusion. At the opposite end of the spectrum, methods that can image the microcirculation directly, such as confocal microscopy, two-photon microscopy and optical coherence tomography, are limited by their penetration or high invasiveness. They can only image microstructures to a depth of a few hundred microns, at best, while keeping the animal alive.

Ultrasound Localization Microscopy (ULM) technology has overcome the compromise between resolution and penetration by allowing deep microcirculation to be visualized. It is based on the monitoring of isolated clinical contrast agents, namely microbubbles, moving through blood vessels and has since been implemented in humans. It is achieved by virtue of the following steps:
injection of contrast agents, often microbubbles, at an optimized concentration,
ultrasound imaging of the region of interest comprising the blood vessels through which the contrast agents flow,
isolation of contrast agents on ultrasound images by virtue of filters that can highlight microbubbles moving at high speed by virtue of spatio-temporal or temporal filters,
subwavelength localization of contrast agents. The principle of super-resolution is herein based on the fact that the microbubbles are separate from each other, preventing interference between their echoes. It is this interference that limits resolution to about half a wavelength. When isolated objects are located, their position can be determined with much greater precision than wavelength based on the signal-to-noise ratio,
monitoring microbubbles to determine their path, and
path accumulation for super-resolution visualization.

The ULM technique is an acoustic super-resolution technique that enables us to map the microcirculation with unprecedented resolution on the scale of the organ of a living animal, which typically results in a gain of a factor of ten.

Although it enables rich microvascular mapping, it remains unsuitable for imaging functional units of organs. This limitation stems from a lack of contrast for these structures. Unlike large vessels, few contrast agents pass through individual microstructures, and they flow very slowly; on state-of-the-art ULM images they therefore do not appear.

The present invention aims to overcome these disadvantages.

The object of the invention is thus a method for ultrasound imaging of a microvascular structure.

The method according to the invention comprises:
a step of injecting ultrasound contrast agents into blood vessels in a region comprising a microvascular structure,
a step of ultrasound imaging said region comprising the microvascular structure while the contrast agents flow through the blood vessels, the concentration of the contrast agents and/or the ultrasound frequency of the imaging being chosen such that the contrast agents are sufficiently separated to prevent feedback,
a step of detecting/filtering and locating the contrast agents,
a step of individually monitoring the contrast agents,
a step of classifying the behaviors of the individually monitored contrast agents according to at least one predetermined behavior characteristic of contrast agents flowing through the microvascular structure, and
a step of imaging the microvascular structure according to the thus-performed classification.

The injection step is typically performed intravenously and the contrast agents are distributed via the circulation.

The imaging step can thus be carried out by selecting and displaying individually monitored contrast agents having a predetermined behavior characteristic of contrast agents flowing through the microvascular structure.

For example, it is known that the contrast agents (microbubbles) visualized travel through the glomeruli, swirling through the capillary balls that form them. The invention consists of translating this very precise behavior, distinct from what is expected in other kidney vessels, to enable classification of single contrast agents flowing through the glomeruli. Filters can be added to categorize contrast agents into those that behave in this way and those that do not. An example of predetermined behavior representative of glomeruli is a stationary behavior of contrast agents over a certain period of time.

Said predetermined behavior characteristic of contrast agents flowing through the microvascular structure may be a predetermined displacement (for example swirling in the case of glomeruli), a predetermined transit time and/or a predetermined speed of the contrast agents.

Said predetermined behavior characteristic of contrast agents flowing through the microvascular structure can also be a variation in the amplitude of the linear or non-linear echo of the contrast agent.

Other predetermined behaviors may for example be a swirling movement of the contrast agents, or other types of movement, changes in their speed, residence time in a restricted region, their mean free path, or even their dispersity.

Using this classification, it is possible to generate a specific image of these microvascular structures, based on trajectories compatible with the predetermined behavior.

This image can then be used to derive fundamental information about these structures, such as their number, their size, their location and their distribution in space.

The microvascular structure can be a functional unit of the region, said region being an organ.

The contrast agents are advantageously separated by a distance at least equal to the ultrasound wavelength.

The contrast agents can be microbubbles.

The microvascular structure may be a glomerulus of a kidney.

In this case, the predetermined behavior characteristic of contrast agents flowing through a glomerulus may be a predetermined displacement, a predetermined transit time and/or a predetermined speed of the contrast agents.

Said predetermined behavior characteristic of contrast agents flowing through a glomerulus may be a predetermined swirling of contrast agents.

The method may comprise:
a step of ultrasound imaging of the kidney, using a device that emits and receives ultrasound and reconstructs an image based on the ultrasound time-of-flight,
a step of injecting ultrasound contrast agents into blood vessels of the kidney,
a step of detecting and locating the contrast agents,
a step of individually monitoring the contrast agents,
a step of detecting contrast agents having a speed greater than a first predetermined speed and of detecting contrast agents having a speed less than a second predetermined speed,
a step of detecting, from contrast agents having a speed less than a second predetermined speed, contrast agents exhibiting a movement characteristic of contrast agents flowing through a glomerulus, and notably a swirling movement,
a step of imaging glomeruli, by selecting contrast agents exhibiting a movement characteristic of a glomerulus.

The step of imaging glomeruli can be carried out by selecting contrast agents exhibiting a characteristic movement of a glomerulus and contrast agents having a speed greater than the first predetermined speed.

The method may further comprise a step of analyzing the number, position and/or variability of contrast agents in the glomeruli.

The object of the invention is also a device for implementing the method.

The device according to the invention comprises:
a system for ultrasound imaging of a region comprising the microvascular structure,
a system for injecting ultrasound contrast agents into blood vessels in said region comprising the microvascular structure,
advantageously, a contrast agent filtering system that does not irreversibly exclude contrast agents according to their speed,
a system for detecting and locating contrast agents,
a system for individually monitoring the contrast agents,
a system capable of imaging the microvascular structure, using ultrasound, and selecting individually monitored contrast agents and having a predetermined behavior characteristic of contrast agents flowing through the microvascular structure.

Further advantages and particularities of the present invention will become apparent from the following description, given as a non-limiting example and made with reference to the attached figures:

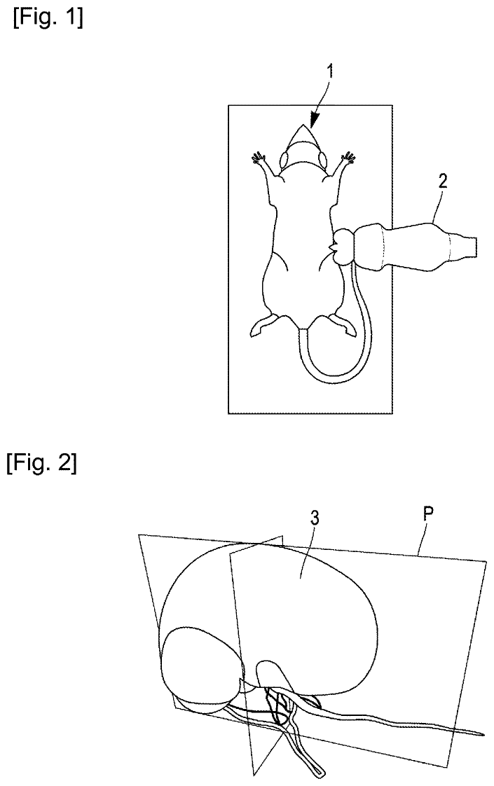

FIG. 1 shows a rat undergoing an imaging method according to the invention,

FIG. 2 shows a kidney of the rat in FIG. 1,

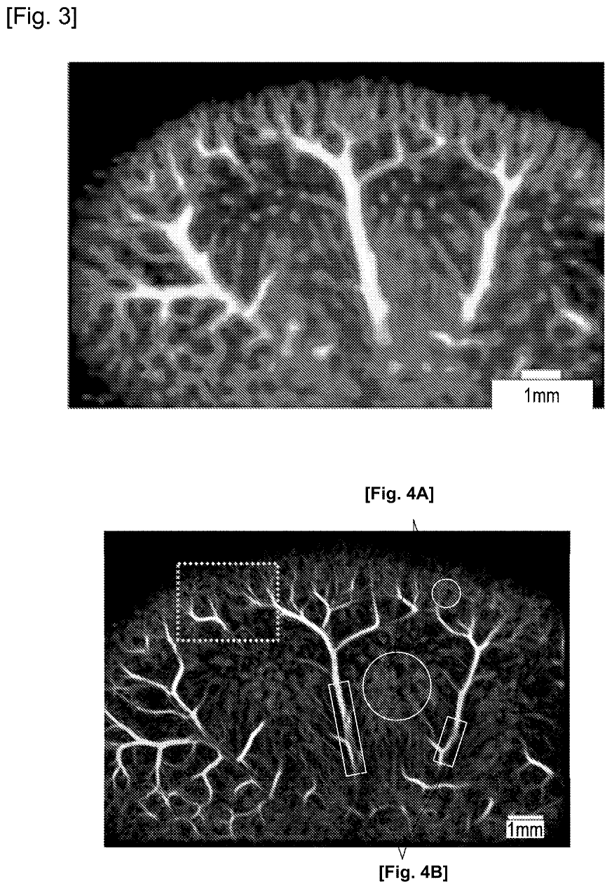

FIG. 3 is a Doppler ultrasound of the kidney in FIG. 2,

FIGS. 4A-4B are images of the kidney, obtained by an imaging method according to the invention,

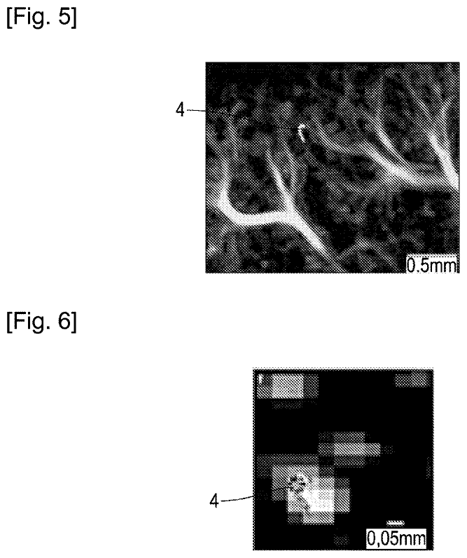

FIG. 5 is a detail view of the image of FIGS. 4A-4B,

FIG. 6 is a detail view of the image of FIG. 5,

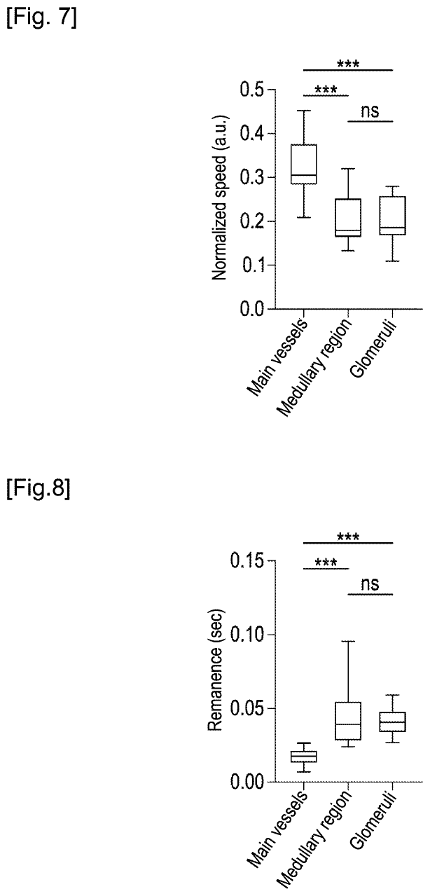

FIG. 7 is a diagram showing the speed of contrast agents in different regions of the kidney of the rat,

FIG. 8 is a diagram showing the remanence of contrast agents in different regions of the kidney of the rat,

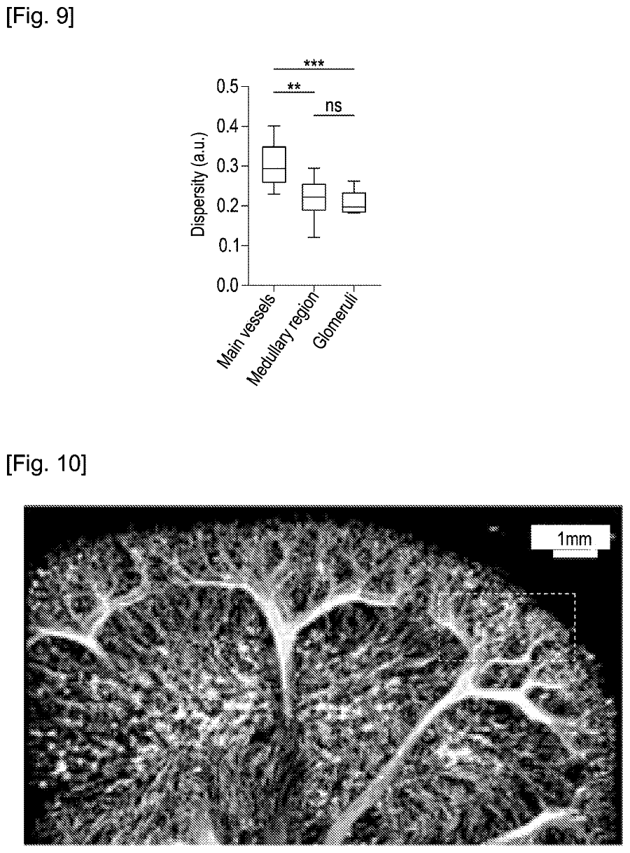

FIG. 9 is a diagram showing the dispersity of contrast agents in different regions of the kidney of the rat,

FIG. 10 is an image of the kidney obtained by ultrasound localization microscopy,

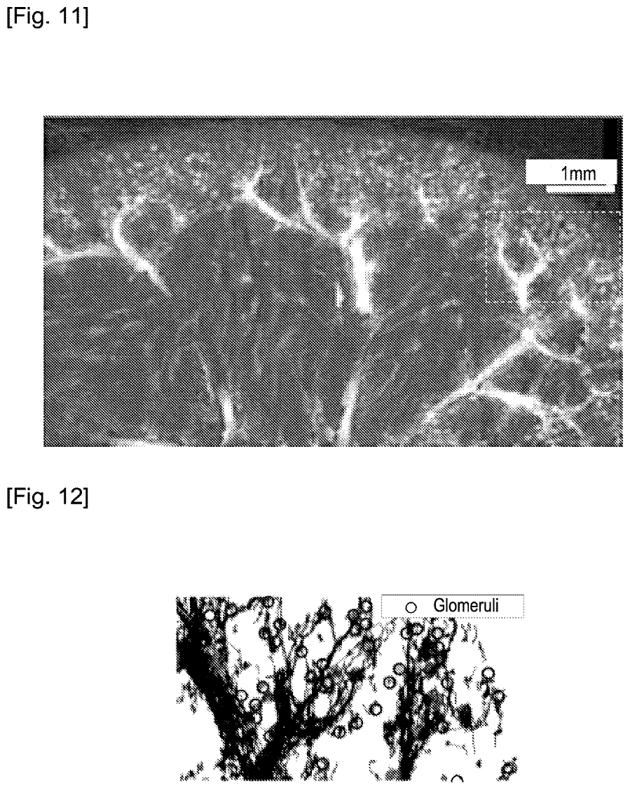

FIG. 11 is an image of the kidney obtained by X-ray microtomography,

FIG. 12 is a detail view of the image of FIG. 10 showing the glomerular count,

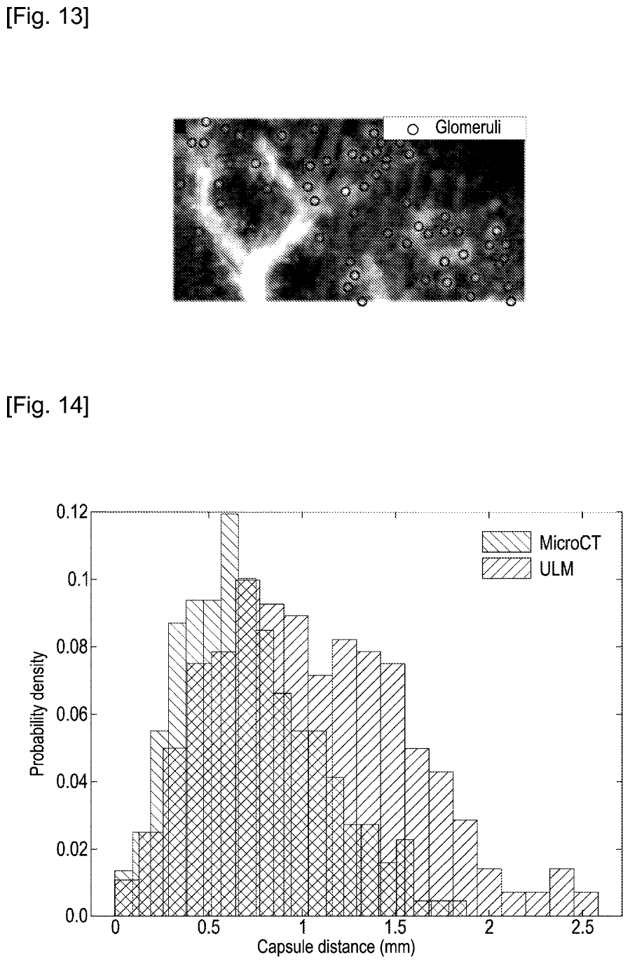

FIG. 13 is a detail view of the image of FIG. 11 showing the glomerular count,

FIG. 14 is a diagram showing the probability of glomerular density,

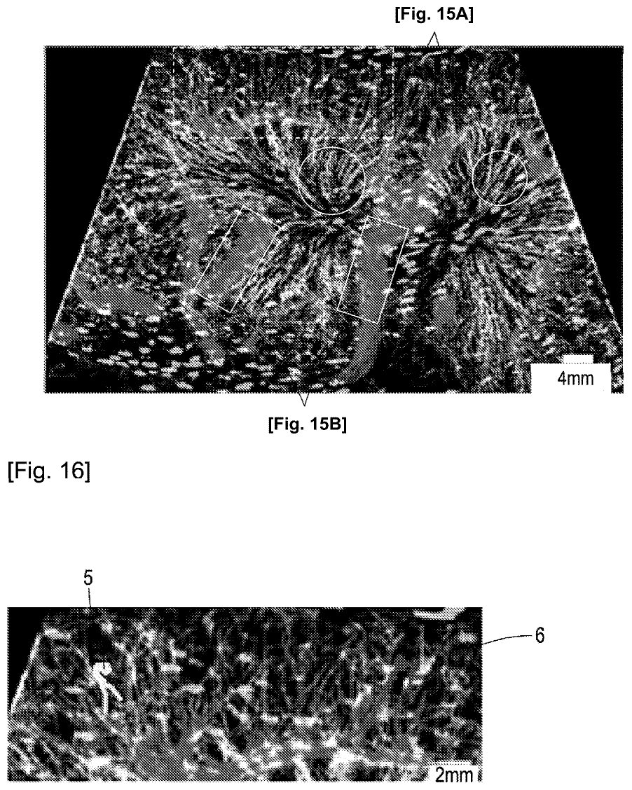

FIGS. 15A-15B are images of a human kidney obtained by an imaging method according to the invention,

FIG. 16 is a detail view of the image of FIGS. 15A-15B,

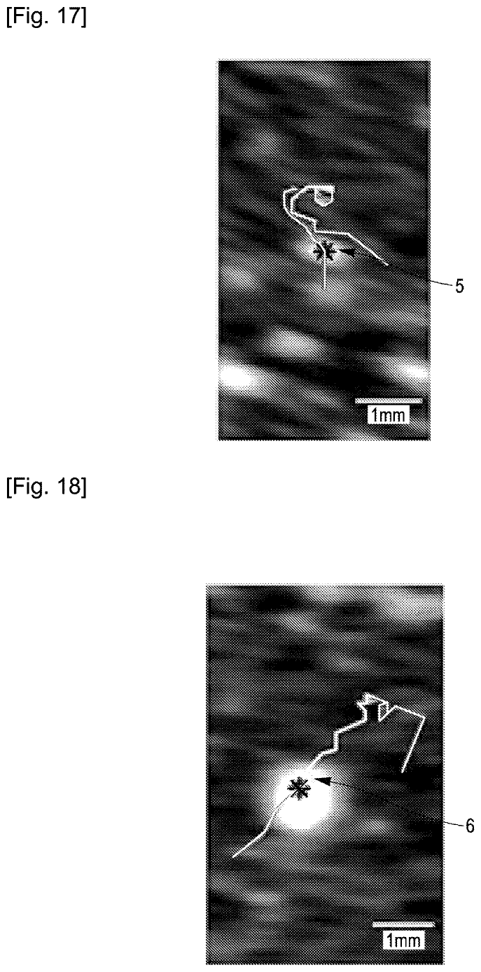

FIG. 17 is a detail view of the image of FIG. 16 showing a first trajectory of a contrast agent in a glomerulus,

FIG. 18 is a detail view of the image of FIG. 16 showing a second trajectory of a contrast agent in a glomerulus,

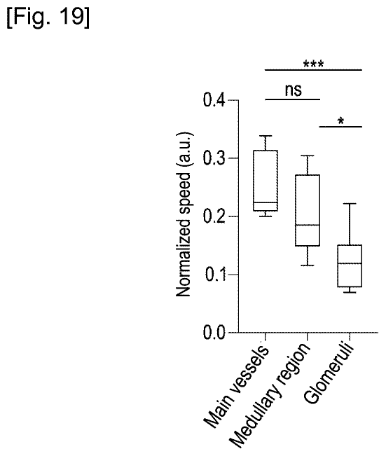

FIG. 19 is a diagram showing the speed of contrast agents in different regions of the human kidney,

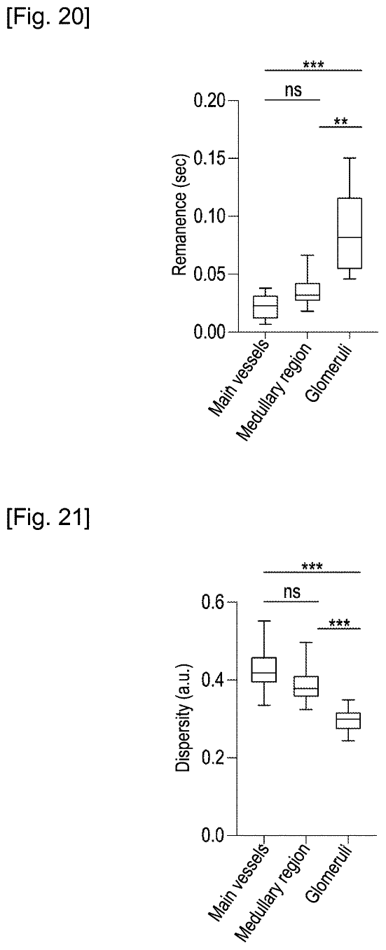

FIG. 20 is a diagram showing the remanence of contrast agents in different regions of the human kidney, and

FIG. 21 is a diagram showing the dispersion of contrast agents in different regions of the human kidney.

### DETAILED DESCRIPTION

### Ultrasound Imaging of Rat Kidney Glomeruli

As a first step, ULM is applied to observe microvessels with low blood flow in a kidney 3 of a rat 1, using a probe 2 (FIGS. 1 and 2). In general, ULM consists of imaging microbubbles in the microvasculature with an ultrasound scanner at an image frequency of 500 Hz and a contrast agent infusion of 13 μL/min, which enable the localization and subwavelength monitoring of individual gaseous particles. Eleven rats were examined using a research ultrasound scanner and a 15 MHz high-frequency linear probe. The animals were placed in a supine position, and the left kidney was externalized to acquire ultrasound data in a P-plane longitudinal to the kidney (FIG. 2). Ultrasound acquisitions consisted of a sequence of long ensemble amplitude (LEAM) in order to obtain conventional linear data, allowing observation of fast microbubbles, and non-linear data to enhance the signal from slow or stopped microbubbles. ULM post-processing steps were then performed twice in order to obtain a fast microbubble monitoring map from the linear acquisition filtered with a singular value decomposition (SVD), and a slow microbubble map from the non-linear acquisition. Conventional Power Doppler ultrasound, that is, performed by virtue of linear acquisition and SVD, is shown in FIG. 3.

By virtue of this double monitoring, it is possible to obtain a composite ULM density map with slow tracks in light gray and fast tracks in dark gray (FIGS. 4A-4B). Two examples of slow-track regions A (inside circles) and fast-track regions B (inside rectangles) are shown in FIGS. 4A-4B and 15A-15B. On this map, we could observe a particular behavior of microbubbles in the cortex: traces of microbubbles entering or leaving a region where they circle at speeds corresponding to the capillary flow. A zoom in on the ULM density map in the cortex (FIG. 5) confirms the presence of these slowly circling ultrasound scatterers. As an example, traces 4 of two independent microbubbles were monitored along this path leading to a glomerular shape (enlarged in FIG. 6): microbubbles in the cortex appear to rise, then swirl before leaving again. Such microbubble behavior can be observed along the intermediate vessels of the cortex.

Segmentation of the different regions of the kidney, namely the medulla, the large arteries (interlobar and arcuate) and the cortex, was carried out manually in order to compare the behavior of microbubbles in the different regions. A glomerular mask was also constructed by selecting cortical tracks exhibiting a high mean free path value, that is, stagnant microbubbles. Tracks which have at least one point in the region were included in the analysis of the region.

Specific metrics to highlight capillary tangles, including normalized speeds, remanence time (that is, the duration of the trace within a 50 micron radius from the center of the trace), and dispersity (that is, the number of times that a trace goes in the same direction, with a tolerance of plus or minus 20°, divided by the number of dots making up the trace) were performed on each trace of the 11 rats. These measurements reveal slower (FIG. 7), more stagnant (FIG. 8) and less dispersed (FIG. 9) microbubbles in the glomerular structures with respect to the traces in the medullary region and in the main arteries. Student's t-tests (with a 5% margin) show that these differences are always significant from the main vessels to the glomeruli (P<0.001 for normalized speed, P<0.001 for remanence, P<0.001 for dispersity), and from the main vessels to the medulla (P<0.001 for normalized speed, P<0.001 for remanence, P<0.002 for dispersity).

The Micro-CT Indicates that the Capillary Tangle Detected in the ULM Corresponds to the Glomeruli

Of the 11 rats examined by ULM in vivo, 6 were also observed post-mortem by micro-CT (X-ray microtomography) after injection of barium contrast medium at the end of ultrasound acquisition. Manual adjustment of the ULM (FIG. 10) and micro-CT slice (FIG. 11) was carried out on a 160 μm slice, that is, corresponding to the elevation resolution of the ultrasound probe at the focal point. Glomerular counting was performed using local high-intensity regional maxima on the micro-CT slice (FIG. 13). On the ULM, capillary tangles were counted by selecting cortical tracks exhibiting a high mean free path value in the ULM (FIG. 12).

Manual segmentation of the renal capsule was performed in order to compare the density of glomeruli based on their distance therefrom, in both imaging modalities. Thus, it can be seen that the spatial distribution is similar between the glomeruli detected by a known approach with micro-CT and the capillary tangles observed by ULM in rat number 7 (FIG. 14). A two-sample Kolmogorov-Smirnov statistical test was performed on the 6 rats with the two modalities in order to determine whether the distributions of the two sets came from the same continuous distribution. The null hypothesis, that is, h, is accepted in all rats, meaning that the glomeruli are equally distributed in both modalities at the 5% significance level.

The size of glomeruli was also calculated in both modalities. For ULM, in 11 rats, the glomerular density curve was plotted based on distance from the center of the glomeruli, and for micro-CT, the intensity curve based on the distance from the central pixel. The full width at half maximum of these normalized data was used to estimate glomerular diameter in all rats. The glomeruli appear to have a similar diameter in both modalities, that is, 60 μm in diameter, and smaller than those observed in the literature, that is, 50 μm in radius.

### Ultrasound Imaging of Human Kidney Glomeruli

ULM was performed in 5 transplant patients who underwent routine clinical ultrasound examination in hospital. Conventional contrast-enhanced ultrasound sequences (CEUS), integrated in a clinical ultrasound scanner and a convex ultrasound probe (2 MHz), were used to acquire clinical data and perform the ULM. The ULM post-processing steps are similar to those carried out in the rat, with the exception that, as the CEUS sequence is a non-linear sequence, the data were split with a bandpass filter to obtain fast microbubbles, and with specific ULM parameters to highlight stagnant microbubbles.

This composite ULM density map, with fast microbubbles in dark gray (regions B) and slow microbubbles in light gray (regions A), shows the same glomerular shapes as rats in the distribution in the cortex (FIGS. 15A-15B). A specific zoom into this region confirms the presence of afferent and efferent arterioles (green) with a swirling bubble in the middle of the path (pink) (FIG. 16). Tracks 5 and 6 are examples of this particular path (highlighted in FIG. 16, and zoomed in on FIGS. 17 and 18).

In the same way as the treatment performed on the rat, the glomeruli were counted and their diameter and their spatial distribution studied using the same technical tools. It was observed that the glomerular diameters estimated by ULM are slightly higher than those found in the literature, that is, 200 μm in diameter, but remain in the same order of magnitude.

Manual segmentations of the regions of the kidney were performed, namely the medulla, cortex and main vessels (interlobar and arcuate arteries), and the glomerular mask was calculated as previously. Thus, it was possible to compare the behavior of the tracks in each region. Student's t-tests were performed with a margin of 5% in 8 patients. In the same way as in rats, and even more pronounced than in humans, glomeruli remain slower than medullary vessels (P≤0.05) and main vessels (P<0.001); they are more remanent, that is, stagnant, than medullary vessels (P≤0.01) and main vessels (P≤0.001); and they are also less dispersed than medullary vessels (P≤0.001) and main vessels (P≤0.001).

### Conclusion

The invention implements a new form of ULM that exploits the kinetics of individual microbubbles, including those flowing through capillaries, to map the microarchitecture of the vessels. By classifying their behavior based on known anatomical characteristics, we are looking in this case for the precise complexion of the glomerular microcirculation, including afferent and efferent arterioles. Rotating microbubbles were monitored specifically in the cortex and further trace analysis confirms the presence of these particular shapes in this region, that is, slower, more stagnant and less dispersed. It was also possible to confirm that what was observed by ULM were glomeruli by virtue of a comparison with micro-CT: the spatial distribution of glomeruli by ULM and by micro-CT statistically belong to the same continuous distribution. In addition, the size of the glomeruli measured by micro-CT and ULM is similar with both techniques in all rats, and close to the results previously found in the literature in rats and humans. Finally, glomeruli are similarly distributed between ULM and micro-CT around intermediate blood vessels.

The method according to the invention therefore enables individual glomeruli in humans to be observed for the first time using a clinical imaging device. Current imaging techniques allow only an indirect estimation of glomerular filtration rate, and not their distinct observation. In addition to being able to observe a new category of slow microbubbles whose behavior and localization are comparable to glomerular organization, the method reduces the gap between anatomical imaging and functional imaging, since it is possible to image the very structures responsible for organ function. By virtue of ULMs and the use of information provided by the microbubble itself, it is possible to image functional microvascular units. This type of imaging can be used to study numerous organs and diseases such as diabetes or cancer. In addition, acquisitions in humans were carried out using a clinical ultrasound scanner that is widely available and used worldwide.

1. A method for ultrasound imaging of a microvascular structure, wherein it comprises:
a step of injecting ultrasound contrast agents into blood vessels in a region comprising a microvascular structure,
a step of ultrasound imaging said region comprising the microvascular structure while the contrast agents flow through the blood vessels, the concentration of the contrast agents and/or the ultrasound frequency of the imaging being chosen such that the contrast agents are sufficiently separated to prevent feedback,
a step of detecting/filtering and locating the contrast agents,
a step of individually monitoring the contrast agents,
a step of classifying the behaviors of the individually monitored contrast agents according to at least one predetermined behavior characteristic of contrast agents flowing through the microvascular structure, and
a step of imaging the microvascular structure, according to the thus-performed classification.

212. The method according to claim 1, wherein the imaging step is carried out by selecting and displaying individually monitored contrast agents having a predetermined behavior characteristic of contrast agents flowing through the microvascular structure.

3. The method according to claim 1, wherein said predetermined behavior characteristic of contrast agents flowing through the microvascular structure is a predetermined displacement, a predetermined transit time and/or a predetermined speed of the contrast agents.

4. The method according to claim 1, wherein said predetermined behavior characteristic of contrast agents flowing through the microvascular structure is a variation in the amplitude of the linear or non-linear echo of the contrast agent.

5. The method according to claim 1, wherein the microvascular structure is a functional unit of the region, said region being an organ.

6. The method according to claim 1, wherein the contrast agents are separated by a distance at least equal to the ultrasound wavelength.

7. The method according to-one claim 1, wherein the contrast agents are microbubbles.

8. The method according to claim 1, wherein the microvascular structure is a glomerulus of a kidney.

9. The method according to claim 8, wherein said predetermined behavior characteristic of contrast agents flowing through a glomerulus is a predetermined displacement, a predetermined transit time and/or a predetermined speed of the contrast agents.

10. The method according to claim 9, wherein said predetermined behavior characteristic of contrast agents flowing through a glomerulus is a predetermined swirling of the contrast agents.

11. The method according to-one of claim 8, wherein it comprises:
a step of ultrasound imaging of the kidney, using a device that emits and receives ultrasound and reconstructs an image based on the ultrasound time-of-flight,
a step of injecting ultrasound contrast agents into blood vessels of the kidney,
a step of detecting/filtering and locating the contrast agents,
a step of individually monitoring the contrast agents,
a step of detecting contrast agents having a speed greater than a first predetermined speed and of detecting contrast agents having a speed less than a second predetermined speed,
a step of detecting, from contrast agents having a speed less than a second predetermined speed, contrast agents exhibiting a movement characteristic of a glomerulus, and notably a swirling movement,
a step of imaging glomeruli, by selecting contrast agents exhibiting a movement characteristic of a glomerulus.

12. The method according to claim 11, wherein the step of imaging the glomeruli is performed by selecting contrast agents exhibiting a movement characteristic of a glomerulus and contrast agents having a speed greater than the first predetermined speed.

13. The method according to claim 11, wherein it further comprises a step of analyzing the number, position and/or variability of contrast agents in the glomeruli.

14. A device for implementing the method according to claim 1, wherein it comprises:
a system for ultrasound imaging of a region comprising the microvascular structure,
a system for injecting ultrasound contrast agents into blood vessels in said region comprising the microvascular structure,
a contrast agent filtering system that does not irreversibly exclude contrast agents according to their speed,
a system for detecting and locating contrast agents,
a system for individually monitoring the contrast agents,
a system capable of imaging the microvascular structure, using ultrasound, and selecting individually monitored contrast agents and having a predetermined behavior characteristic of contrast agents flowing through said microvascular structure.
